(19) United States
(12) Patent Application Publication
(10) Pub. No.: US 2015/0196432 A1
(43) Pub. Date: Jul. 16, 2015

(54) Ventilated Disposable Garment

(71) Applicant: Chanda Renee' Browning, Conyers, GA (US)

(72) Inventor: Chanda Renee' Browning, Conyers, GA (US)

(21) Appl. No.: 14/153,044

(22) Filed: Jan. 12, 2014

(51) Int. Cl. A61F 13/493

(52) U.S. Cl. CPC A61F 13/493; A61F 2013/49098

(57) ABSTRACT

The present invention is a disposable diaper of conventional construction that has centrally located apertures in the front and/or rear of the garment. The apertures size and contour allows for an adequate amount of air to flow throughout the diaper covered region reducing or eliminating diaper rash. The apertures contain an outer fold or flap that can be sealed and resealed as desired by the user. The inner layer of dry weave or interlocking material forms a leak proof barrier underneath the flap or cover to prevent leakage.

### CROSS-REFERENCE TO RELATED APPLICATIONS

U.S. Pat. No. 2,544,069 Ventilated Infant's Garment

### Inventor: Henry H. Cutler (Lakeside, Mich.)

### Current U.S. Class: 604/385.25

Publication date: 1951 Mar. 6
U.S. Pat. No. 4,887,602 Ventilated Diaper or Incontinent Garment

### Inventor: Audrey A. O'Leary (Belfair, Wash.)

### Current U.S. Class: 604/385.25; 604/389

Publication date: 1989 Dec. 19
U.S. Pat. No. 5,263,948 Breathable Disposable Diapers

### Inventor: Hamzeh Karami (Mansfield, Mass.) & Ronald E. Vitaris

### Current U.S. Class: 604/383; 604/358; 604/385.21

Publication date: 1993 Nov. 23

### BACKGROUND OF INVENTION

The present invention relates to a disposable infant or adult garment, and more particularly, to a disposable diaper with centralized apertures in the front and/or rear of the diaper. The apertures are fashioned to provide ventilation to an individual's diaper-covered area, reducing or eliminating diaper rash.

A conventional disposable diaper consists of an absorbent pad positioned between two sheets of nonwoven fabric. The pad is specifically designed to absorb and retain bodily fluids while the nonwoven fabric provides comfort and helps prevent leakage. The single most important property of a diaper, cloth or disposable, is optimal absorbency. Absorbance and moisture creates the environment for diaper rash.

Since diapers are close-fitting, this results in inadequate ventilation allowing minimal or no air to flow through the diaper covered area. The skin becomes warmer and moister, creating a higher pH level than other areas of the body. Excessive moisture on the skin makes it more easily penetrable by irritants and more vulnerable to swelling, thus causing diaper rash.

Formally known as diaper dermatitis, diaper rash is any type of skin eruption characterized by swelling, redness or chafing of the area that is covered by the diaper. Although the problem is usually minor, it can, if ignored, lead to the widespread infection of bacteria or fungi requiring aggressive and prolonged treatment.

There are several variations of disposable diapers that claim to provide ventilation; however, the proven effectiveness and success of a ventilated diaper decreasing or eliminating diaper rash is largely unknown at this time. As an answer to the problem above, this invention has larger sized, centralized apertures that allow a considerable amount of air to flow throughout the diaper covered regions.

### BRIEF SUMMARY OF THE INVENTION

This invention is related to an improvement in the structure of a disposable diaper. The primary object of the present invention is to provide an improved disposable diaper which has better ventilation.

Having a proper amount of airflow is important for the healing process of a diaper rash and for the prevention of perspiration in the diaper covered area. A general and important object of the present invention, therefore, is to provide a unique disposable diaper which addresses the issues above in a practical and satisfactory manner, and which offers another significant advancement in the field of infant and/or adult care.

According to a preferred embodiment of the invention, a garment is proposed which includes a diaper, on the front and/or rear sides of which is formed horizontal and rectangular shaped apertures that allows air to flow in and out of the garment. There is a layer of dry weave or similar interlocking material positioned directly behind the apertures to prevent fluid or waste from leaking out of the garment.

The advantages of such a garment are: reduction or elimination of diaper rash, preventing excess moisture from building up in the skin, and giving adequate ventilation to the diaper covered area. These and other important features and advantages that are offered by the invention will become more apparent as the description is read in conjunction with the accompanying drawings.

### BRIEF DESCRIPTION OF THE SEVERAL VIEWS OF THE DRAWINGS

FIG. 1 is a plan view of a ventilated disposable diaper constructed in accordance with the present invention. The sealed frontal aperture [1] is centrally positioned and when opened, allows an adequate amount of air to pass throughout the diaper covered region.

FIG. 2 is a plan view, like FIG. 1, except it shows the inner layer of the rear aperture which is comprised of a dry-weave or similar interlocking material [2] that is placed directly behind the aperture. This material prevents the leakage of bodily fluids. The rear aperture is centrally located like that of the frontal aperture.

FIG. 3 is a plan view of the present invention; it emphasizes the inside layer of dry weave or interlocking material of the frontal aperture as it is open or unsealed [3]. FIG. 3 also shows the adhesive like material located on the inside (at the bottom) of the frontal aperture [4] that allows the aperture to be sealed down or dosed.

FIG. 4 is a plan view of the present invention. Each aperture has a horizontal, rectangular shape. The outer layer only, which becomes the cover or flap when opened, is cut out on three sides—bottom [5], left side[6] and right side[7]—and the top portion of the aperture remains attached to the outer layer of the diaper[8] forming a cover or flap for sealing and unsealing.

### DETAILED DESCRIPTION OF THE INVENTION

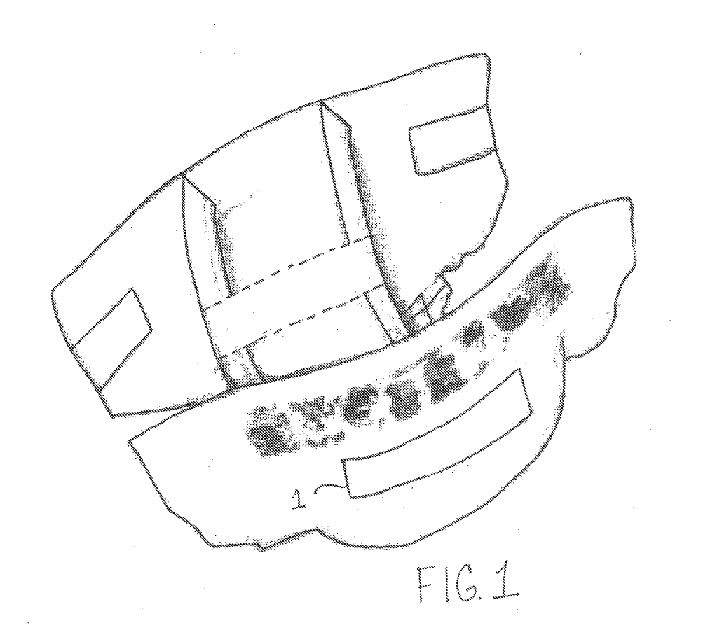

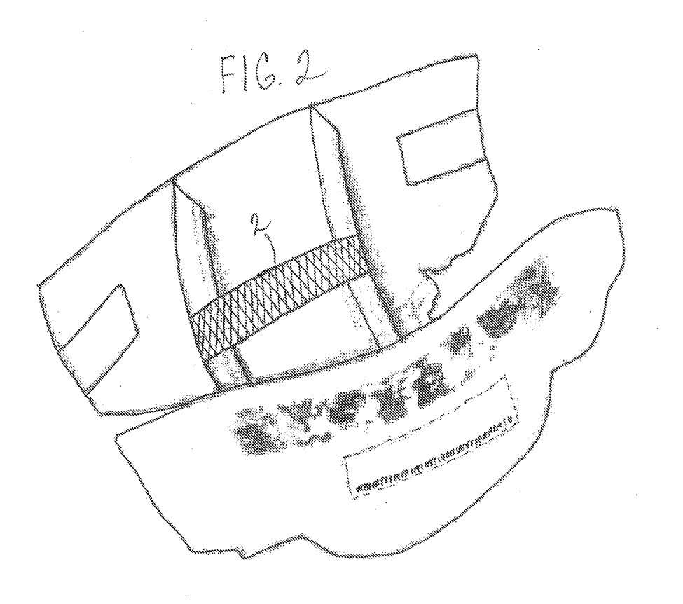

For the purpose of understanding the elements of the invention, reference will be made to the embodiment illustrated in the drawings. In reference to the drawings and in particular to FIGS. 1 and 2 thereof, the diaper according to the present invention generally comprises a conventional disposable diaper. The diaper contains a horizontal shaped aperture, centrally located in the front and/or rear below the elastic waistband on the front and rear sides.

The apertures contour allows for a large amount of air to flow throughout the diaper covered region. The air passes through a dry weave or similar interlocked ventilated material allowing the excessive moisture to be released. The dry weave like material is attached to the inside of each aperture, serving as a barrier between the individual wearing the disposable diaper and the outside of the outer layer.

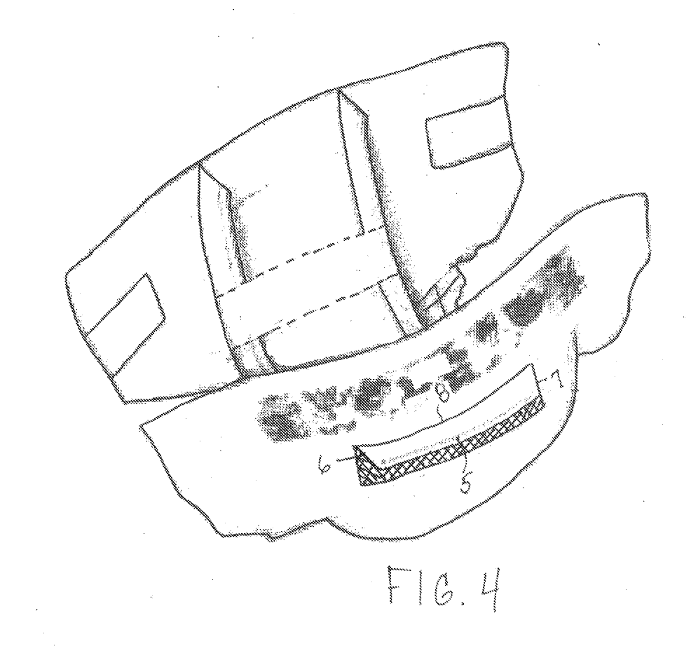

The apertures penetrate through all layers of the diaper, including the inner layer padding and outer layer nonwoven fabric so that a complete ventilation system is formed. As in FIG. 4, each aperture has a layer of material detached on three sides yet remains attached to the top and forms a cover or flap that can be opened and closed at will. The aperture covers or flaps are made of the exact material as the outermost layer of the diaper.

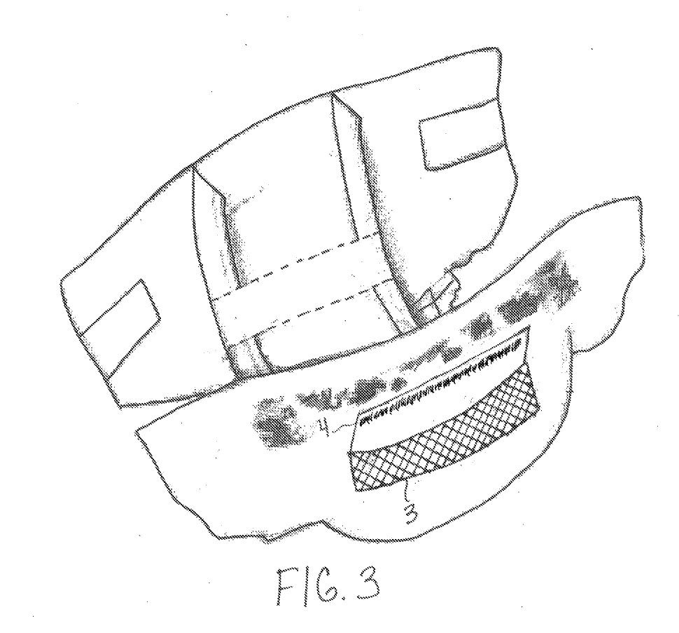

As in FIG. 3, each aperture contains a thin layer of adhesive material positioned across the length of the bottom on the inside and on the outside of the cover or flap.

I claim:

1. A disposable diaper comprising one or more ventilating apertures, centrally located in the front and/or rear of the diaper in an area below the waistband.

2. The garment of claim 1, contains horizontal and rectangular shaped apertures with a length ranging between about 3 and 100 mm.

3. The garment of claim 1, contains horizontal and rectangular shaped apertures with a width ranging between about 2 and 50 mm.

4. The apertures of claim 2, contains a cover or flap that is permanently attached, across the top, to the outside layer of the disposable diaper with the ability to be sealed and unsealed.

5. The apertures of claim 2, contains a dry weave type or interlocking material directly attached to the inside layer, forming a barrier that prevents leakage when the flap is opened.

6. The garment of claim 1, contains a flap or covering over the apertures that contain a thin layer of adhesive material across the length and on the inside and outside of each of them.
